(19) United States
(12) Patent Application Publication
(10) Pub. No.: US 2005/0113661 A1
(43) Pub. Date: May 26, 2005

(54) EKG recording accessory system (EKG RAS)

(76) Inventors: Alireza Nazeri, St. Claire Shores, MI (US); Iraj Nazeri, St. Claire Shores, MI (US)

Correspondence Address: DYKAS, SHAVER & NIPPER, LLP, PO BOX 877, BOISE, ID, 83701-0877, US

(21) Appl. No.: 10/911,995

(22) Filed: Aug. 4, 2004

Related U.S. Application Data
(63) Continuation-in-part of application No. 10/719,604, filed on Nov. 21, 2003.

(51) Int. Cl. A61B 5/04

(52) U.S. Cl. 600/386; 600/391; 600/393

(57) ABSTRACT

The invention is a precordial pad for positioning EKG electrodes on a patient for anatomically correct and repeatable placement. Data can be transmitted from the EKG pad of the invention by wire or wireless means. The pad includes a sizing aid and a positioning device. The invention is also a system for obtaining and sending EKG data.

### PRIORITY

This application is a Continuation in Part of the utility application entitled EKG Recording Accessory System (EKG RAS) filed by Alireza Nazeri on Nov. 21, 2003, with Ser. No. 10/719,604.

### BACKGROUND OF THE INVENTION

1. Field of the Invention

The present invention generally relates to an EKG contact electrode pad, and more particularly relates to EKG contact pads with temperature sensors, sizing selection and placing means.

2. Background Information

Electrocardiography (EKG, ECG) is a medical test for recording the electrical activity of the heart. In the standard twelve lead EKG there are twelve (12) different wires that carry electrical signals from the area of the body to which they are attached. Certain leads are attached to the person's chest in six standard areas. These are known as precordial leads. Four of the twelve leads are the four limb electrodes: right wrist, left wrist, right ankle and left ankle. In some cases, the placing of two extra lead electrodes in the right side of the patient's chest allows the possibility to record the EKG of the right heart. The limb leads are designated RL, RF, LL and LF, and attach respectively to the two ankles and the two wrists of the patient. The precordial leads are designated as V₁, V₂, V₃, V₄, V₅ and V₆, and the leads for the right side of the heart are designated VR₁ and VR₂. The limb leads can be placed in an “adjusted” position, rather than on the extremities. The adjusted position for the limb leads is on the torso of the patient.

From the time of the invention of the EKG to present usage, each electrode is generally connected separately to the EKG recorder by wire. This means that for the routine twelve lead EKG, we need at least ten (10) separate electrodes attached to standard anatomical positions and ten (10) wires that go separately to the EKG machine. In the configurations including the right heart EKG, they will become twelve (12) separate electrodes. These standard electrode placements can also be used for electrodes for an external pacemaker, a defibrillation device and for real time heart monitoring of the patients in critical care units.

The results of the EKG will be printed as a graph on standard paper or shown on the monitor. EKG is the most commonly used diagnostic test in medicine for evaluating the function of the heart. Reading the EKG is very important in patient management as the difference between a normal and an abnormal reading can be measured in millimeters on the chart. Correct placement of electrodes in the standard positions, attachment to the skin, perfect conductivity, and the least artifacts as possible in the recording are the keys in the repeatability, accuracy and reliability of this procedure. For the best performance, a skilled physician or technician should place the electrodes. With the currently available methods of electrode placement, there can be significant errors produced in the EKG recordings. For example, one person may place the electrodes in a different position than another person, and the same person can place them in another position at a different time. Even if placed predictably, it could be placed in a wrong anatomical position.

Thus, in the conventional placement of the electrodes, the repeatability, accuracy and reliability of the data are suspect, especially in emergency situations when procedures are carried out rapidly and in difficult situations.

Therefore, what are needed are repeatability, consistency and accuracy in the placement of electrodes for an EKG recording on the same patient with different users or on different patients by the same user.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

The EKG pad of the invention accomplishes these objects, as well as others. The repeatability is provided by an array of electrodes that are mounted in a disposable precordial pad. The precordial pad of the invention has an array of EKG electrodes and a temperature sensor distributed on a flexible, multi-layer material. The flexible material forms a pad body, which has an outer surface and a body surface. The body surface of the precordial pad body includes an adhesive layer, which stabilizes the pad on a person's torso and ensures that the readings are taken during a test from one, and only one position on the patient's chest. The adhesive layer of the precordial pad is covered until it is ready for use. The precordial pad is covered by an adhesive cover, which is stripped off to expose the adhesive surface when the pad is ready to be used. The body surface also includes conductive electrodes and a temperature sensor, which will contact the patient's skin.

The pad also has a middle layer, which is the main circuit layer. The circuit layer includes a printed circuit that collects all data from all applicable electrodes, sensors and attachments of the pad and brings them to the one area on the outer surface of the pad. The circuit layer is designed and made to be capable of tolerating higher voltages that may be used for defibrillation. The printed circuit can be made of copper, conductive ink or other electrically conductive material.

The outer surface pad also includes a signal export area. This is where the data-transmitting module attaches to the pad and carries all of the signals from one point to the EKG machine. The signal export module can be a wireless transmitter that transmits data from the precordial pad to the EKG machine via the system adaptor. The module is also capable of transmitting data via a conventional wiring harness, as a single cable includes a bundle of wires leaving the main precordial pad to a designed universal adaptor of the invention, which is connected to an EKG machine.

The invention is also a system for taking the EKG of a patient, which has a capability of performing defibrillation, external pacing and monitoring the patient's heart at the same time and with the same precordial pad. The system includes a disposable EKG precordial pad as described above, as well as some additional components. The additional components include a measuring device, which is used to measure the size of the patient's test area such as his chest or torso. Depending on the size of the patient's test area, a size of precordial pad is selected based on the testing system of the invention. For instance, sizes 1, 2, 3 and 4 may be available for various sizes of patients. Sizing can also include consideration for gender, as pads for males are likely to be larger than those for females. Pad sizes can also be designated as Small, Medium, Large, Extra Large, etc. Other designations are obviously possible and would be related to an indication on the measuring device of the invention. The correct size of the pad can also be determined based on the patient's gender and shirt size.

The system includes a positioning device, which can be used to measure a patient for pad size, as well as aid the caregiver in positioning a pad correctly on a patient. The positioning device of the system is used with the well-known anatomical marker on the human chest called the Supra Sternal Notch. By placing a curved edge of the positioning device on the patient's supra sternal notch, the precordial pad can be placed accurately and consistently in the anatomically correct position, with the electrodes thus placed correctly. This feature allows non-professional users to place the EKG electrodes on themselves with high accuracy. This has not been possible with EKG electrodes in the prior art.

The pad is also composed of materials that are to be translucent to the X-Ray so patients can wear the pad while they are being x-rayed. The pad is also designed and composed of materials that are to be water resistant and waterproof. The pad is also made from biocompatible material to have the least allergic reaction for the patients. The pad may also be worn while the patient is getting an MRI.

The Signal Export Module is another part of this system. This has an interface for connection to the signal export area of the pad. The signal export module receives signals from the related electrodes and sensors. It can include a connection site for connection of a single cable, which can be used to transmit the data to the universal adaptor. The cable can be regular wire or fiber optic. The module can also contain a micro-transmitter to transmit data wirelessly to the universal adaptor. This will have the benefit of wireless transmission and can utilize bluetooth, infrared, wi-fi or other wireless technologies. One way to select between wireless and wired transmission is to activate the wireless mode, unless a cable is connected to the module. It would typically have a rechargeable long life lithium battery or another suitable battery type. The signal export module can also have a data recognition sensor to sense the EKG signals and send an alarm if the patient has certain preprogrammed changes in his or her EKG, such as arrhythmias. The wireless feature of the pad allows the patient to wear the pad, put the module on wireless mode and be able to move around, go to the bathroom, go to the lunchroom, move in a wheelchair, etc. The pattern recognition ability of the system will automatically send an alarm signal if an abnormal event happens.

The Universal Adaptor/Receiver is another part of the invention. Its features will include compatibility with all of the current or future EKG recorders in the market. It includes an input site for the wires from the recorder and a site for connection of the wires from the pad and limb electrodes. This part will be used for the wire transmission of data from the pad to the EKG machine. The adaptor/receiver also contains a receiver for receiving data wirelessly from the micro-transmitter and transferring them to the recorder. It also includes a digital display to show body temperature. A switch will allow the adaptor/receiver to select wire or wireless transmission mode, and to change output to the selected format for the EKG machine in use, or for a defibrillator, external heart pacing system or for real time monitoring.

The pad is disposable so that it will be used for only one patient. This will limit the risk of transmitting skin disorders from one person to another, which is a concern in the currently available method.

An important feature of the pad is that the electrodes embedded in the pad extend from the pad surface for better contact. Rather than being flush with the pad, the electrode layer of the pad includes a device that causes the electrodes to extend away from the pad by two to five millimeters. The electrode-extending device would also exert a small amount of pressure so that when the pad is attached to the patient's chest, the extended electrodes press harder against the patient's skin than they would otherwise. The electrode extension device can be some type of biased device, such as a coiled spring or some other type of spring. The electrode extension device can also be a biased member made of foam. The foam structure would be compressed under the electrode when the adhesive cover is applied. When the adhesive cover is removed, the compressed foam would force the electrode to extend out from the body surface of the pad by two to five millimeters or more, preferably.

A foam pad or other biased device would also apply the correct pressure that would be transmitted to the electrode and thus, to the patient's skin. This will produce the highest quality contact and conductivity, which is directly related to the performance of the recording.

The body surface of the pad includes an adhesive layer made from biocompatible and non-allergic materials. This will be attached to the skin upon removal of the cover. Another feature of the invention is that the electrodes may be pre-coated with a transmitting gel, which would be sandwiched between the electrode and the cover of the adhesive layer. When the adhesive cover is removed, the transmitting gel would remain on the electrode contact surface and be available to improve the connection between the electrode and the patient's skin. All of these features result in a precordial pad that can improve the repeatability of test results, which can stabilize the pad during a particular test, which can read low temperatures and send that information to the EKG machine, and which facilitates rapid, accurate and repeatable placement of the precordial pad of the invention. This pre-application of gel also eliminates a possible route of cross contamination.

The precordial pad of the invention also includes a temperature sensor built into the pad body. The temperature sensor measures a low range of body temperatures. It is when a patient's body temperature is in a low range that the electrical pattern of the heart will be affected. Knowing this factor in the recording is key to distinguishing the pattern of a normal from an abnormal EKG, as an EKG taken from a patient who is at a below normal temperature will have altered the readings. If that EKG is reviewed at a later time, a full interpretation of the EKG readings would not be possible without knowledge of the patient's temperature at the time the reading was taken. For that reason, a temperature sensor is built into the pad body. The temperature sensor would also be linked to the data-transmitting module and sent to the EKG machine for recording with other data.

The micro-transmitter for the limb electrodes uses the same technology for the four electrodes of the limb leads. This can be associated with each single electrode for wireless transmission, if applicable.

Added features of the precordial pad of the invention are connection points to the four limb electrodes. These sensing sites are on the four limbs of the patient, including the right arm, left arm, the right ankle and the left ankle or their adjusted positions on the chest of the patient. The designed sets of limb electrodes of this invention are also capable of attachment on the chest, rather than on the limb to simplify the installation of electrodes for the EKG test if the user chooses. The pad body of the invention would include sites to allow electrodes from the four limbs to connect to the pad body and be routed with the information from the other electrodes of the pad body to the EKG machine.

Further, the purpose of the foregoing abstract is to enable the United States Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

Still other objects and advantages of the present invention will become readily apparent to those skilled in this art from the following detailed description, wherein I have shown and described only the preferred embodiment of the invention, simply by way of illustration of the best mode contemplated by carrying out my invention. As will be realized, the invention is capable of modifications in various obvious respects all without departing from the invention. Accordingly, the drawings and description of the preferred embodiment are to be regarded as illustrative in nature, and not as restrictive in nature.

### BRIEF DESCRIPTION OF THE DRAWINGS

FIG. 1 is a view of the surface of the precordial pad, which would face away from the patient.

FIG. 2 is a view of the circuit layer of the precordial pad, with detachable limb leads.

FIG. 3 is a view of the circuit layer of the precordial pad with detachable limb leads in the attached position.

FIG. 4 is a view of the precordial pad with limb leads attached, showing the surface that contacts the patient.

FIG. 5 is a view of the precordial pad that does not have limb leads, showing the side that contacts the patient.

FIG. 6 is a view of the attachable limb leads.

FIG. 7 is a view of a positioning device detached from the pad.

FIG. 8 is a view of the electrodes in closed and opened positions.

FIG. 9 shows various configurations of the universal adaptor.

FIG. 10 shows the internal structure of the universal adaptor.

FIG. 11 is a view of the data-transmitting module.

FIG. 12 shows the pad of the invention with optional attachment to an EKG machine and selected non-EKG devices.

FIG. 13 is a diagram showing anatomical markers and electrode positions used in the invention.

FIG. 14 shows a front and profile view of human anatomy showing anatomical markers and electrode positions.

FIG. 15 is a drawing that shows patients of different sizes, in comparison with a chart that shows different sizes of EKG device that would be used with different sizes of patients.

FIG. 16 shows a number of alternative configurations of the EKG pad of the invention in use with different patients.

### DESCRIPTION OF THE PREFERRED EMBODIMENTS

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

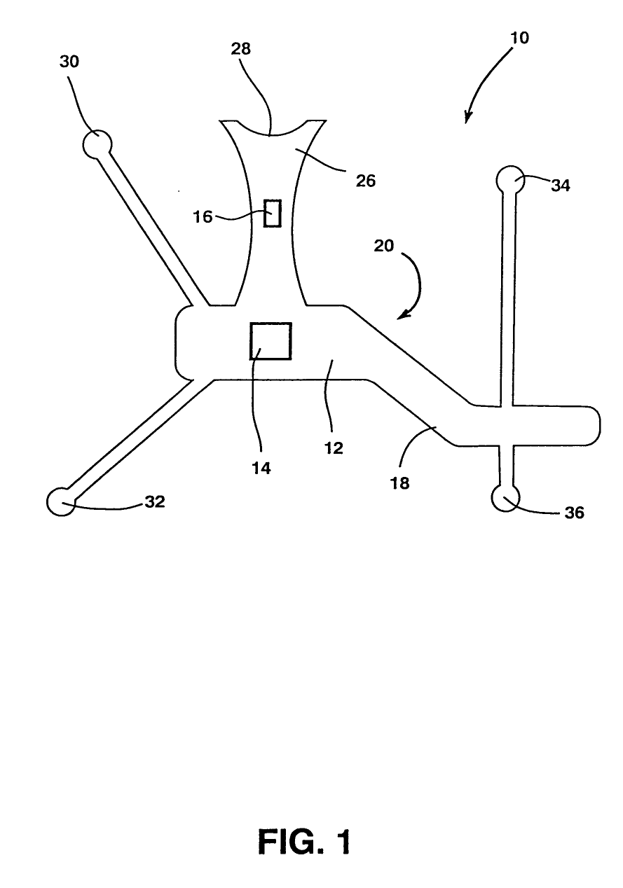

Several preferred embodiments of the invention are shown in FIGS. 1-12. FIG. 1 shows a disposal EKG precordial pad of the invention, which is designated as 10. This embodiment of the invention includes a pad body 12, which includes a sliding site for module attachment 14 and a temperature window 16. The pad body includes an outer or first surface 18 and a body or second surface 20. FIG. 1 is a view of the outer surface, with the body surface 20 being located on the opposite side of this view of the pad body 12. In this view of the precordial pad 10, the embedded electrodes are not visible. A data-transmitting module or signal export device 22 interfaces with the sliding site for module attachment 14. This will be discussed further in other figures. A temperature sensor 24 is also present in the device, with data from the temperature sensor 24 being displayed in the temperature window 16. The precordial pad 10 includes a positioning extension 26. In the embodiment shown in FIG. 1, the positioning extension 26 is attached to the pad body 12. Other embodiments of the device could include a positioning extension 26 that is detachable or not attached at all to the pad body 12.

A doctor, technician or any professional or non-professional user uses the positioning extension 26 on a patient to determine the correct placement of the precoridal pad 10. The positioning extension 26 includes a supra sternal notch 28. The supra sternal notch 28 is meant to be placed adjacent the manubrium, which is the bone adjacent to the jugular notch directly above the ribcage and at a patient's throat. By placing the supra sternal notch 28 of the precordial pad 10 adjacent the jugular notch of the patient, the electrodes of the precordial pad are assured of being placed in the proper anatomical position on a patient. Also included in the precordial pad 10, shown in FIG. 1, is an upper right limb lead 30. Although this is on the left hand side of FIG. 1, it would be associated with the patient's right side. Also, in the embodiment in FIG. 1 are a lower right limb lead 32, an upper left limb lead 34 and a lower left limb lead 36.

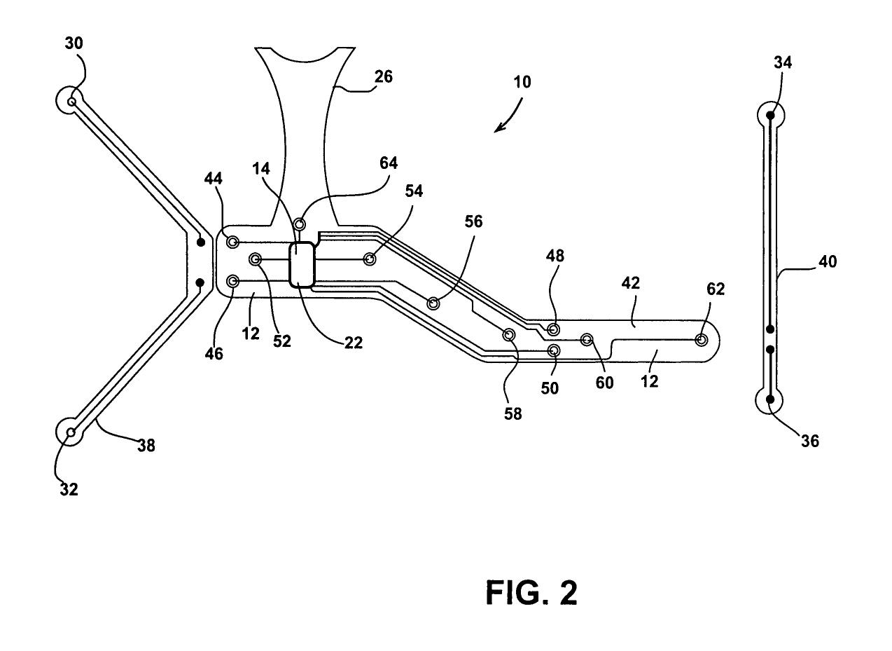

Shown in FIG. 2 is another favored embodiment of the invention. In this embodiment, the limb leads are available as attachments to the pad body 12. This embodiment includes a right limb lead assembly 38 and a left limb lead assembly 40. The embodiment shown in FIG. 2 shows the circuit layer 42 of the pad body 12. In the circuit layer 42, the electrical connections that are associated with each electrode are visible. The electrodes include an upper right limb lead connection 44 and a lower right limb lead connection 46 to which the upper right limb lead 30 and the lower right limb lead 32 are connected when the right limb lead assembly 38 is attached to the pad body 12. The electrical connections would be sufficient to carry higher voltages if it were to be used with a defibrillation option. In such a case, only certain predetermined electrodes would be used for defibrillation.

Similarly, an upper left limb lead connection 48 is provided as well as a lower left limb lead connection 50. These are provided so that a connection can be made with the upper left limb lead 34 and the lower left limb lead 36, which are part of the left limb lead assembly 40. These limb lead assemblies 38 and 40 can optionally be snapped into place or the pad body may be used without limb leads. Electrode 52 is the V₁ electrode, electrode 54 is the V₂ electrode, the electrode 56 is the V₃ electrode, electrode 58 is the V₄ electrode, electrode 60 is the V₅ electrode, and electrode 62 is the V₆ electrode. The positions of these electrodes, V₁ through V₆, correspond to known electrode geometries and provide an accurate EKG reading when positioned on the patient's body correctly.

As in FIG. 1, the embodiment of FIG. 2 includes a positioning extension 26. As can be seen in FIG. 2, electrical connections between each of the electrodes are made with the module attachment site 14. A data-transmitting module 22, not shown in FIG. 2, is utilized to transmit the data from each of the electrodes to the EKG machine. Electrode 64 is provided to obtain a temperature reading, which is conveyed to the site for module attachment 14 and to the data-transmitting module 22.

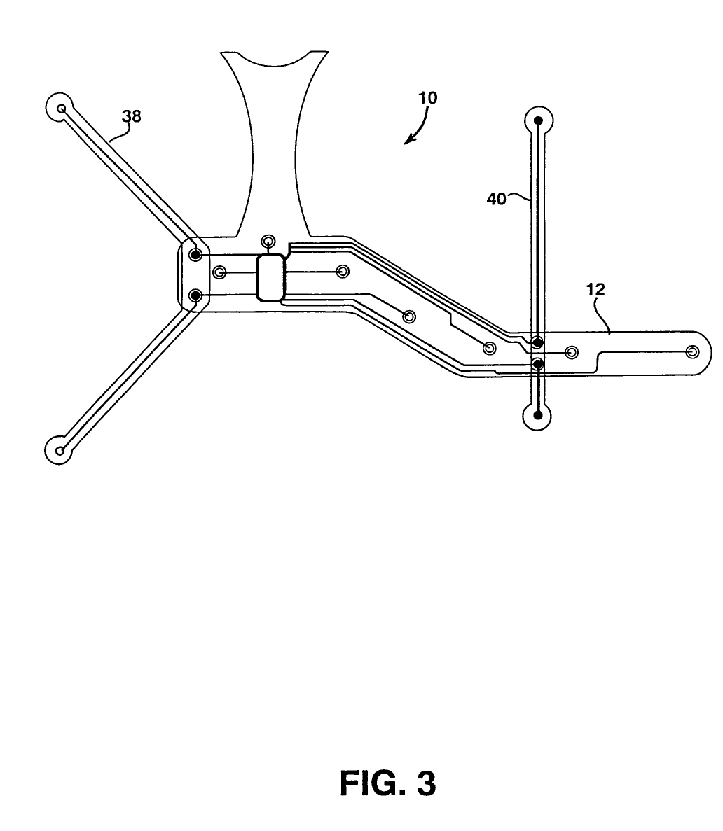

FIG. 3 shows the right limb lead assembly 38 and the left limb lead assembly 40 attached in place on the pad body 12, showing the circuit layer 42.

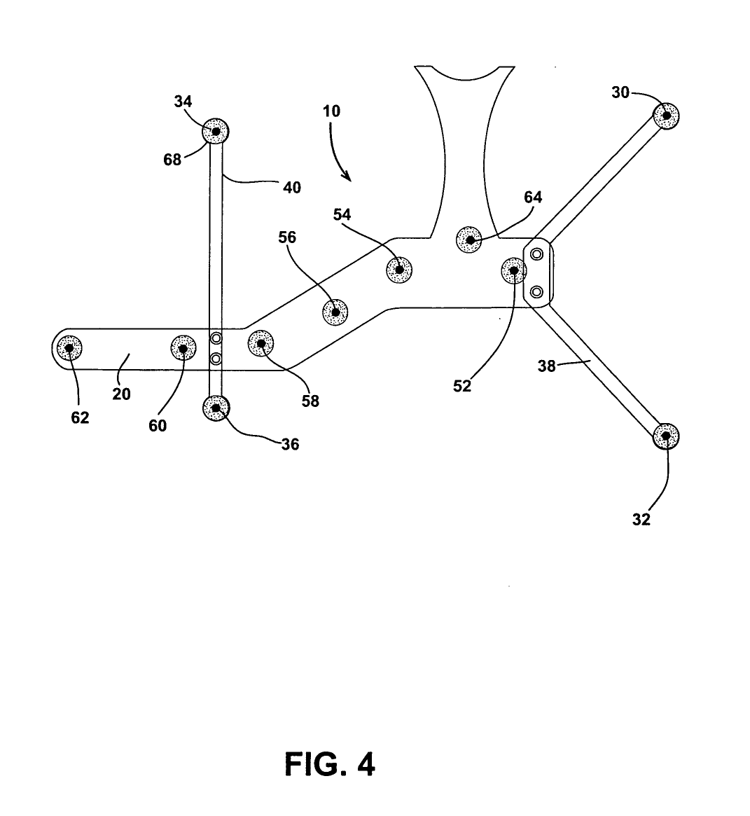

The embodiment shown in FIG. 4 is the same as that in FIG. 3. However, what is shown is the body surface of the pad body, also called the body surface layer. This is the view of the device as it would contact the patient's body. The electrodes 52, 54, 56, 58, 60, 62 and 64 are shown. They are connected by the electrical connection shown in FIG. 3, which is not visible in this view. The right and left limb lead assemblies 38 and 40 are shown in their attached configuration, attached to the connections 44, 46, 48 and 50. Shown around each electrode is a zone of adhesive material. Adhesive material may also optionally be placed on the pad body 12 in various locations.

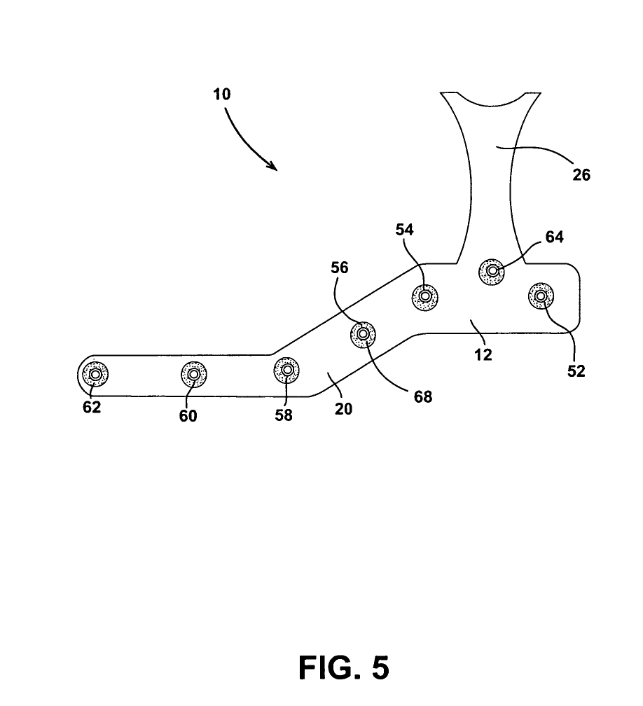

FIG. 5 is a view of the second surface 20 of the pad body, the surface that contacts the patient's skin. This version of the device does not have the right or left limb lead assembly and shows an optional configuration of the precordial pad 10.

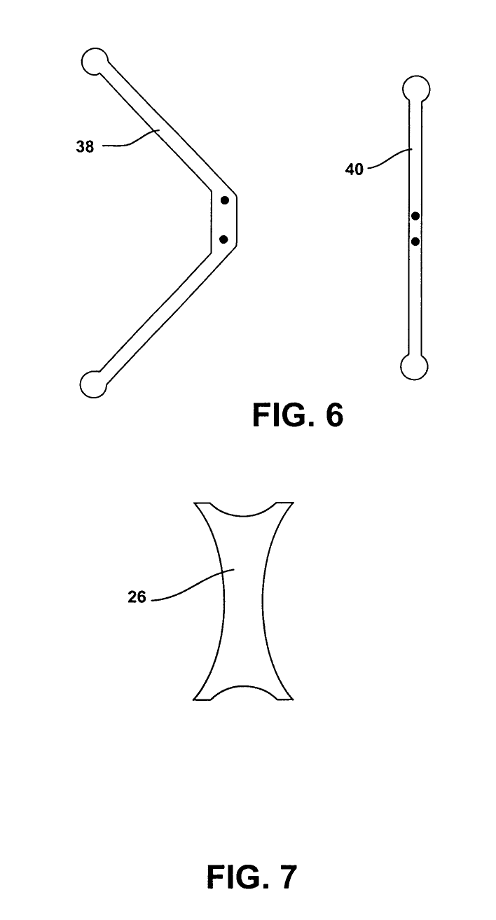

FIG. 6 shows a view of the right limb lead assembly 38 and the left limb lead assembly 40, which may be optionally used with the versions of the precordial pad 10 which are shown in FIGS. 2-4.

FIG. 7 shows the positioning extension 26 which can be detachable from or used as a separate piece with the precordial pad 10.

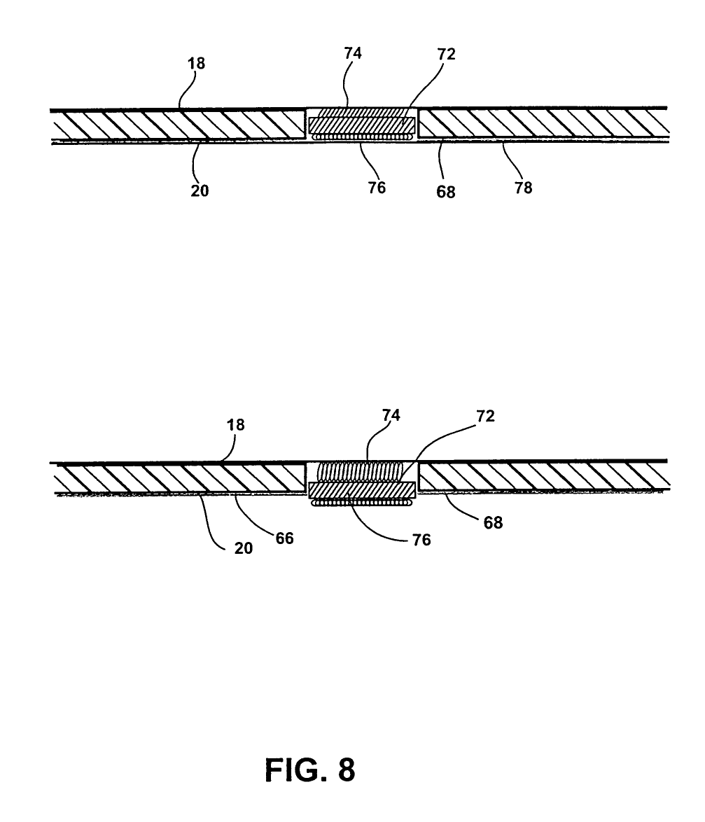

FIG. 8 shows a cross-sectional and enlarged view of an electrode 72 in the precordial pad. Also shown is the first surface of the pad body 18 and the second surface of the pad body 20. The second surface of the pad body 20 would be positioned against the skin of the patient. Between the electrode 72 and the first surface 18 is a biased member 74. The biased member 74 is a device that is stored under some degree of compression and when released, expands and causes the electrodes 72 to move away from the first surface 18. The biased member 74 can be a spring, such as a coil spring, or it can be a compressible substance such as foam. When released, either the spring or the foam would expand and cause the electrode 72 to move away from the first surface 18. On the electrode 72, the surface opposite the biased member 74 is a conductive gel 76. The conductive gel 76 is added to the surface of the electrode 72 during manufacture. On the second surface 20, a layer of adhesive 68 is located. A cover layer 78 covers the adhesive 68. When the cover layer 78 is removed, as shown in the lower corner of FIG. 8, the biased member 74 expands and pushes the electrode 72 away from the first surface 18. Removal of the cover layer 78 exposes the adhesive surface 68 and the gel 76.

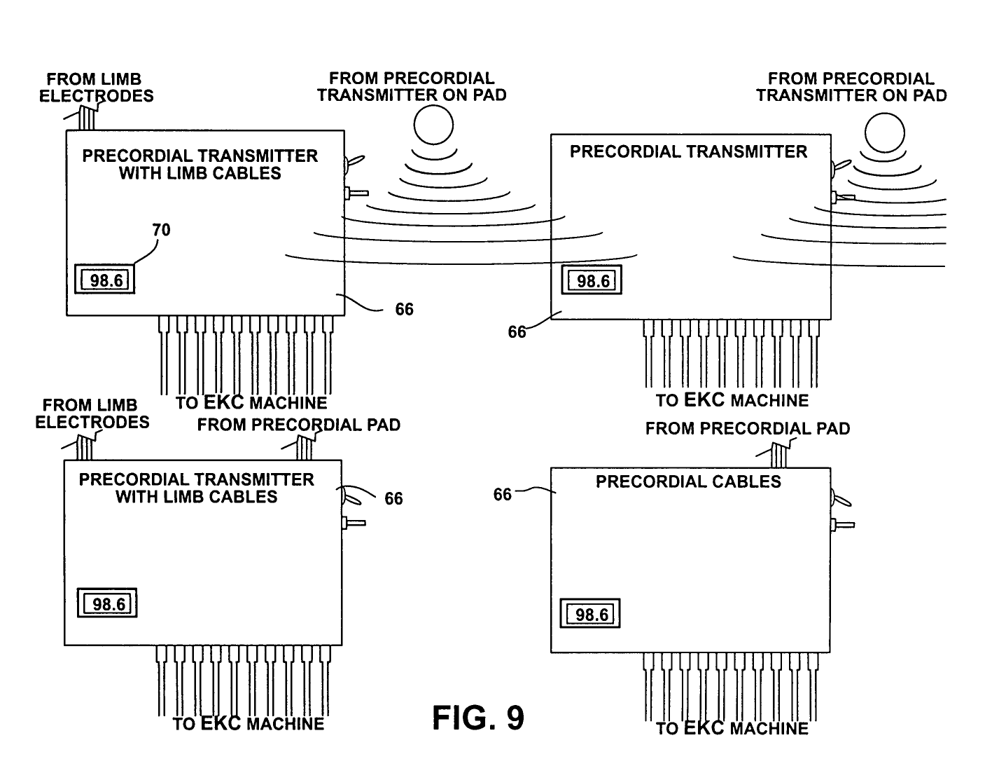

FIG. 9 shows a number of configurations by which the EKG system of the invention would transmit information to any EKG machine. Shown in FIG. 9 is the universal adaptor/receiver of the accessory system. The universal adaptor/receiver is numbered 66. The universal adaptor can take several configurations, which are shown in FIG. 9. In the upper left corner of FIG. 9 is an example of the adaptor/receiver 66 of the invention configured for wireless reception of information from electrodes from the precordial pad. It is also configured for hardwired input of data from the limb electrodes. Shown on the adaptor/receiver 66 is a temperature window 70, which is a separate window from the temperature window 16, which is located on the precordial pad. From the adaptor/receiver 66, wires extend to the EKG machine. From the foregoing description, it will be apparent that various changes may be made without departing from the spirit and scope of the invention as defined by the following claims.

In the lower left corner of FIG. 9 is a depiction of a universal adaptor/receiver 66 of the invention, which is configured for hardwire transmission of data from the precordial pad and from the limb electrodes.

In the upper right corner of FIG. 9 is a depiction of the adaptor/receiver 66, which is configured to receive wireless transmission from both the electrodes of the precordial pad and limb electrodes.

In the lower right corner of FIG. 9 is a universal adaptor/receiver 66 configured to receive hardwired information from the electrodes of the precordial pad and wireless data from the limb electrodes. Any of these configurations of the universal adaptor/receiver 66 of the invention are possible.

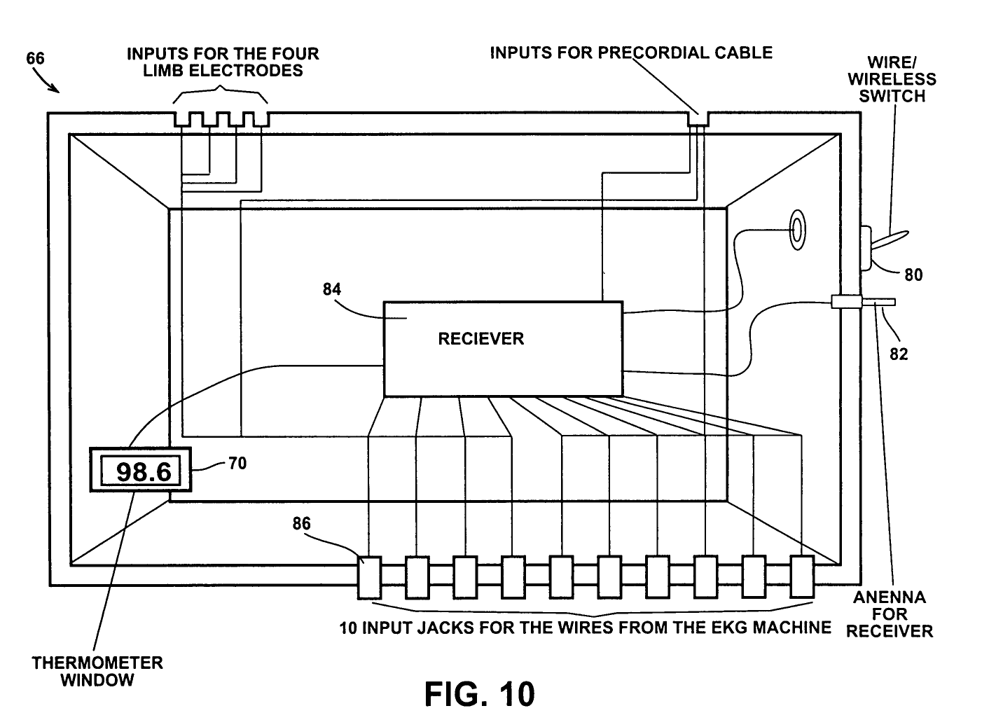

FIG. 10 is a view of some of the details of the universal adaptor/receiver 66. Shown, are inputs for the four limb electrodes as well as inputs for the precordial cable. A wireless switch 80 is shown for switching the unit from wireless to wired operation. Also shown is an antenna 82 for receiving a wireless signal from the precordial pad of the invention. The antenna 82 is connected to a receiver 84 that receives, processes and transmits the information from the precordial pad to outlet jacks 85. Outlet jacks 85 are available for connection to the EKG machine. This would typically be by a wired connection, but using wireless technology for this connection would also be possible. A thermometer window 70 is also shown.

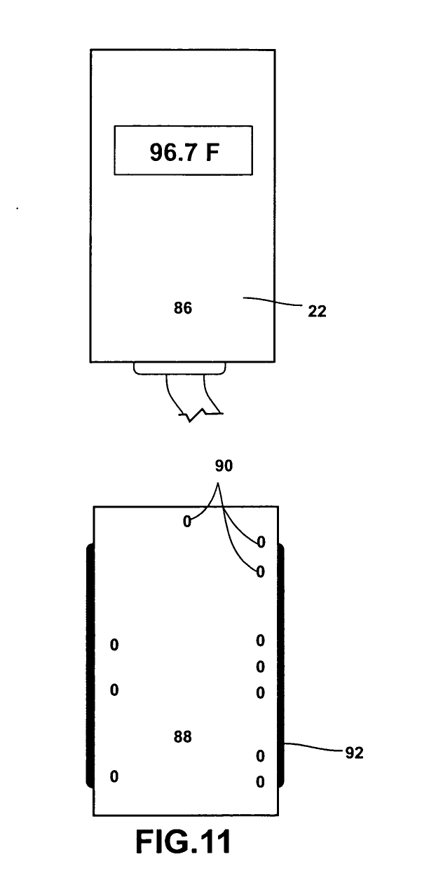

FIG. 11 shows a system-transmitting module, which has also been called the signal export device 22. It has a first surface 86 and a second surface 88. The second surface 88 includes contact points 90 which provide electrical connection with the electrodes or the precordial pad. The signal export device 22 connects to the precordial pad 10 by means of the sliding site for module attachment 14. The signal export devices include sliding borders 92, which allow it to slide into a positive engagement with the sliding site for module attachment 14. Although brackets on the side of the unit are shown, attachment could be accomplished by a number of configurations, as are well known in the industry. This unit could be operated with a cable 94 or could operate by wireless transmission.

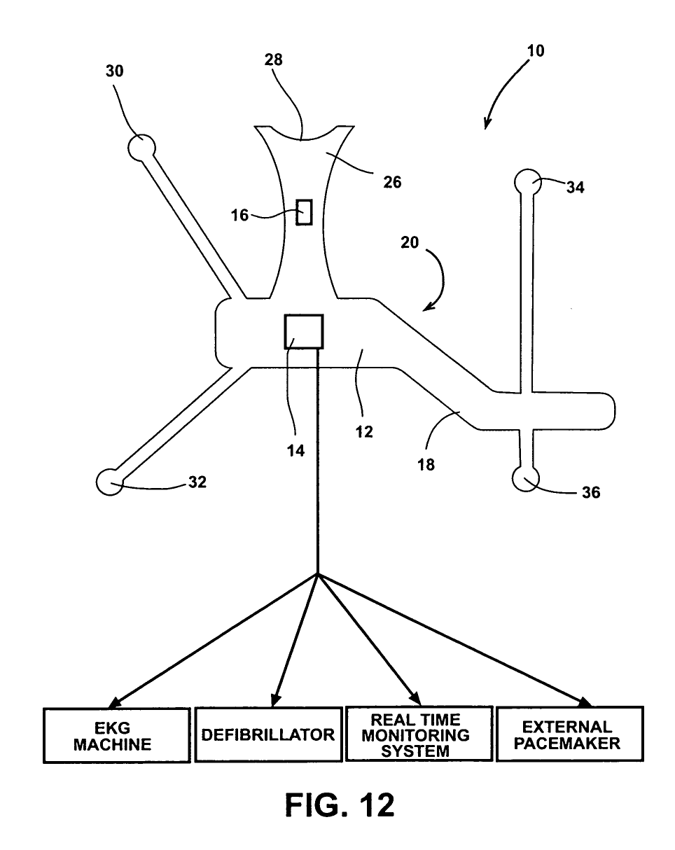

FIG. 12 shows a pad of the invention and possible connections with which it can be used. These include an EKG machine, a defibrillator, a real time heart monitoring system and an external heart-pacing machine.

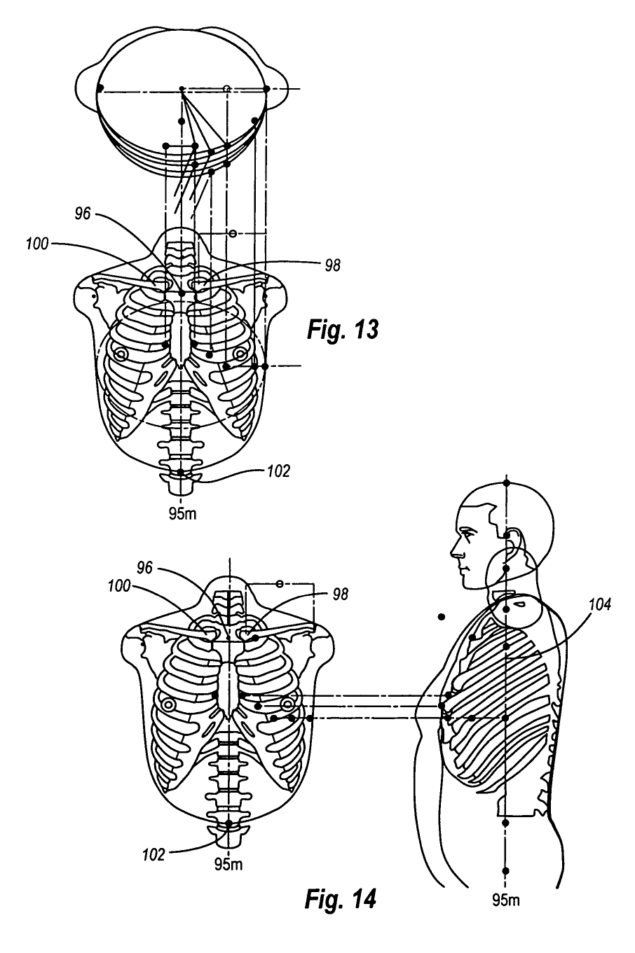

FIG. 13 shows the correct anatomical position for EKG electrodes, and certain anatomical features that are used to position one preferred embodiment of the EKG pad of the invention. Shown in FIG. 13 is the supra sternal notch 96, and adjacent to that are the left and right sternal clavicular joints 98 and 100. Shown at 102 is the umbilicus or belly button.

FIG. 14 shows the same anatomical features along with a profile view of a patient, showing where the electrodes are placed in relation to the lateral line 104 of the patient.

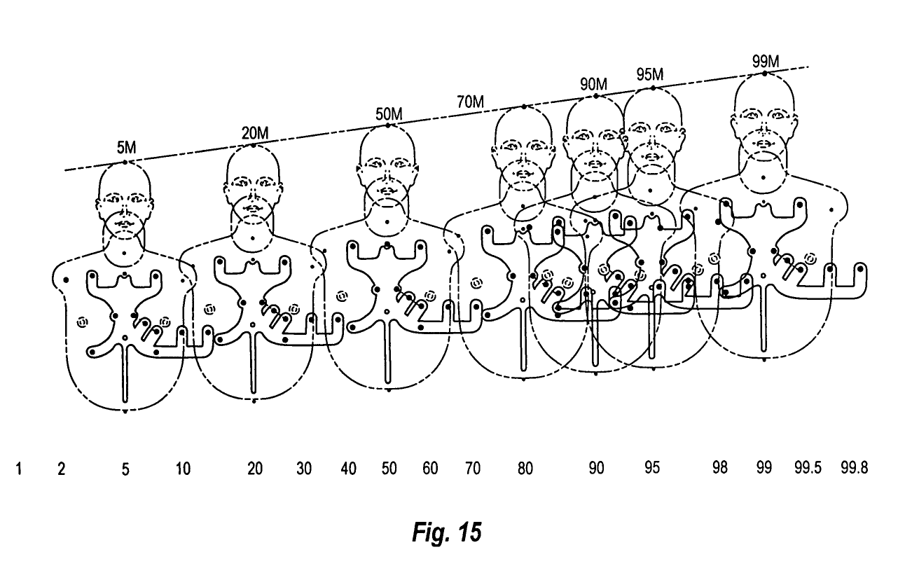

FIG. 15 shows an alternative configuration of the EKG pad along with the use of this pad in placement on different sizes of patients. In FIG. 15, the numbers along the bottom of the chart show the percentile values of different sizes of patients. For instance, the patient at the fiftieth percentile would have about fifty percent of the population smaller than he or she, and fifty percent of the population larger than he or she. The person at the ninety-ninth percentile would find that ninety-nine percent of the population is smaller than that person. This chart illustrates the way in which the EKG pad of the invention adapts to various sized patients, that is by having proportional pads of different sizes. For instance, on each of these pads, the electrodes are in fixed positions but the different sizes of pads can be selected in order to properly fit any size of patient.

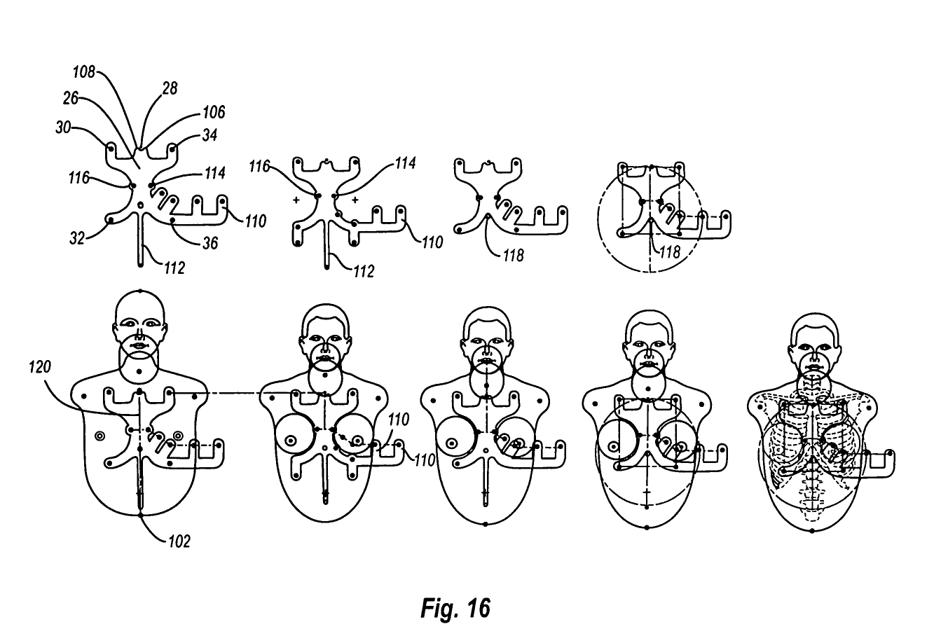

FIG. 16 shows four different preferred configurations of the EKG pad of the invention, and how that particular configuration would be utilized on a patient. The configuration furthest to the left shows a positioning extension 26, which has a supra sternal or manubrium notch 28, which lines up with the super sternal notch of the patient. Adjacent to the manubrium notch 28 is located a left clavicular joint lobe 106, which is a lobe of the position extension which is placed over the left sternal clavicular joint of the patient. On the right side of the manubrium notch 28 is located a right clavicular joint lobe 108 for covering the right sternal clavicular joint of the patient. Also shown are upper right limbs lead 30, lower right limbs lead 32, upper left limbs lead 34 and lower left limbs lead 36. Also shown are six EKG leads with four of them on extensions 110. The extensions 110 allow the electrodes to be placed over three dimensional topography variations of the patient. Also shown is an additional positioning device, which is the umbilical extension 112. The umbilical extension 112 extends in the same vertical line as the manubrium notch 28, and it is either placed over or in line with the umbilicus 102 of the patient. By aligning the precordial pad 10 of the invention between the supra sternal notch 96 and the umbilicus 102, the EKG pad of the invention is guaranteed that it is aligned with the medial line 120 of a patient. Placement of the left and right clavicular joint lobes over the sternal clavicular joints of the patient assures that the precordial pad is placed in an anatomically correct position in the horizontal plane. This version of the precordial pad also includes a left positioning arc 114 and a right positioning arc 116. These are curves that are defined by the pad body and are useful for the placement around topological features on a patient's torso.

The EKG pad, second from the left in FIG. 16, has many features that are shown in the figure to its left. It also has an umbilical extension 112, extensions 110 on which electrodes are placed and a left and right positioning arc 114 and 116. The figure below that embodiment shows how it would be used with the female patient. This configuration allows the electrodes to be placed in an anatomically correct position with the left and right positioning arcs 114 and 116 between the breasts of a female patient. The extensions 110 facilitate placement of the pad body on the patient's torso, with the electrodes on the extensions 110 being able to be placed in the anatomically correct position on the patient's side or on the side of a breast. The EKG pads, third and fourth from the left, shown in FIG. 16 are other variations of the precordial pad of the invention. These do not have an umbilical extension 112, but they do have an umbilical alignment notch 118. This notch allows the precordial pad 10 to be aligned so that it is lined up with the medial line 120 of the patient's body.

While there is shown and described the present preferred embodiment of the invention, it is to be distinctly understood that this invention is not limited thereto but may be variously embodied to practice within the scope of the following claims.

1. A disposable EKG precordial pad comprising:
a pad body for conductive attachment to a patient's torso, with a first and a second surface, with a plurality of electrodes for contact with a patient, and with an adhesive surface on said body surface for contact with said patient's skin; and
a positioning device for use with a patient for orienting and positioning said pad body for correct positioning on said patient, said positioning device utilizing an upper anatomical feature located on the patient's mid line, and a lower anatomical feature located on the patient's mid line, in order to achieve correct placement of the pad body along two axes.

2. The disposable EKG precordial pad of claim 1, which further includes electrodes that are embedded in said pad body but which extend from said pad body before application for electrical connection to the patient's torso.

3. The disposable EKG precordial pad of claim 2, which further includes a data-transmitting module for sending a plurality of signals from said embedded electrodes to an EKG machine.

4. The disposable EKG precordial pad of claim 1 which further includes a temperature sensor for sensing the temperature of said patient's skin, and for sending temperature information to said EKG machine.

5. The precordial pad of claim 1 in which the data-transmitting module is a wireless transmitter.

6. The precordial pad of claim 1 in which the data-transmitting module is a wire cable with individual wires.

7. The disposable EKG precordial pad of claim 1 in which said plurality of electrodes includes one or more micro-transmitters for sending a signal from said electrode.

8. The disposable EKG precordial pad of claim 1 which further includes a circuit layer which is located between said first and said second layer, and which includes electronic connections between said electrodes and said data transmitting module.

9. The disposable EKG precordial pad of claim 8 in which said circuit layer is comprised of an insulating sheet on which is placed electrical connections in the form of conductive pathways.

10. The disposable EKG precordial pad of claim 9 in which said conductive pathways are metallic ink circuitry.

11. The precordial pad of claim 1 which includes an adhesive cover that is removable for exposing said adhesive surface before use.

12. The precordial pad of claim 1 in which said electrodes include an electrode extension device for causing said electrodes to extend beyond said adhesive surface when said adhesive cover is removed.

13. The precordial pad of claim 12 in which said electrode extension device is a biased member mounted between said electrode and said pad, which is held in biased position by said adhesive cover, and which moves said electrode away from said pad when said adhesive cover is removed.

14. The precordial pad of claim 12 in which said electrode extension device is a spring.

15. The precordial pad of claim 12 in which said electrode extension device member is a foam structure.

16. The precordial pad of claim 1 in which said electrode is packaged under said adhesive cover with a layer of transmitting gel so that when said adhesive cover is removed, said transmitting get and said embedded electrodes are configured to be exposed.

17. The precordial pad of claim 1 in which said thermometer sensor is a low reading thermometer sensor.

18. The precordial pad of claim 1 that further comprises six precordial electrodes in a predetermined geometry.

19. The precordial pad of claim 18, which further includes connection points for attachment of limb electrodes.

20. The precordial pad of claim 19, which further includes connection points for attachment of four limb electrodes at the user's discretion.

21. The precordial pad of claim 1 that further comprises six torso electrodes and four limb electrodes built into a single precordial pad.

22. The precordial pad of claim 18, which further includes two additional electrodes for right heart monitoring.

23. The precordial pad of claim 1 in which said embedded electrodes each have a micro-transmitter for wireless transmission of a signal to said data transmitting module.

24. The precordial pad of claim 1 in which said signal export module further includes a temperature display for indicating the patient's body temperature.

25. The precordial pad of claim 1 in which the signal export module is a wireless transmitter.

26. The precordial pad of claim 1 in which the data-transmitting module is a wire harness for connection to an EKG machine.

27. The precordial pad of claim 1 that further includes a low temperature window for displaying patient temperature information.

28. The precordial pad of claim 1 in which said pad further includes capability to connect to non-EKG devices including defibrillators, real time heart monitors and external pacemakers.

29. An EKG recording accessory system, which comprises:
a selection of disposable precordial pads in different sizes, said precordial pads with a plurality of electrodes and with attached positioning devices for use in accurately positioning said pad;
a universal adaptor/receiver which is compatible for connection to all EKG recording and monitoring machines, for receiving data from said electrodes and transferring data to an EKG machine and said precordial pad;
with said positioning device utilizing an upper anatomical feature located on the patient's mid line, and a lower anatomical feature located on the patient's mid line, in order to achieve correct placement of the pad body along two axes.

30. The EKG recording system of claim 29 in which said universal adaptor/receiver further includes capability to connect to non-EKG devices including defibrillators, real time heart monitors and external pacemakers.

31. The EKG recording accessory system of claim 29 in which said precordial pads of said system further include a temperature sensor in a pad body for measuring said patient's skin temperature.

32. The EKG recording accessory system of claim 29, in which said universal adaptor/receiver of said system further includes a temperature reading window.

33. The EKG recording accessory system of claim 29 in which said universal adaptor/receiver of said system further includes a switch to select between wireless and hardwired operation.

34. The EKG recording accessory system of claim 29 in which said disposable precordial pads of said system comprise:
a pad body for conductive attachment to a patient's torso with a first and a second surface, with an adhesive surface on said body surface for contact with said patient's skin;
a plurality of embedded electrodes in said pad body which extends from said pad body for electrical connection to the patient's torso;
a data-transmitting module for sending a plurality of signals from said embedded electrodes to an EKG machine; and
a temperature sensor for sensing the temperature of said patient's skin, and for sending temperature information to said EKG machine.

35. The EKG recording accessory system of claim 34 in which said data transmitting module is a wireless transmitter.

36. The EKG recording accessory system of claim 34 in which said data transmitting module is a wire cable with individual wires.

37. The EKG recording accessory system of claim 34 in which said plurality of electrodes includes one or more micro-transmitters for sending a signal from said electrode.

38. The EKG recording accessory system of claim 34 in which said disposable precordial pad further includes a circuit layer which is located between said first and said second layer, and which includes electronic connections between said electrodes and said data transmitting module.

39. The EKG recording accessory system of claim 38 in which said circuit layer is comprised of an insulating sheet on which is placed electrical connections in the form of conductive pathways.

40. The EKG recording accessory system of claim 39 in which said conductive pathways are in the form of metallic ink circuitry.

41. The EKG recording accessory system of claim 34 in which said disposable precordial pads of the system further include an adhesive cover, which is removable for exposing said adhesive surface before use.

42. The EKG recording accessory system of claim 41 in which said embedded electrodes of said disposable precordial pads of the system include an electrode extension device for causing said electrodes to extend beyond said adhesive surface when said adhesive cover is removed.

43. The precordial pad of claim 42 in which said electrode extension device is a biased member mounted between said embedded electrode and said pad, which is held in biased position by said adhesive cover, and which moves said electrode away from said pad when said adhesive cover is removed.

44. The EKG recording accessory system of claim 43 in which said electrode extension device is a spring or spring like device.

45. The EKG recording accessory system of claim 44 in which said electrode extension device member is a foam structure.

46. The EKG recording accessory system of claim 43 in which said embedded electrode is packaged under said adhesive cover with a layer of transmitting gel in which said transmitting gel and said embedded electrodes are exposed when said adhesive cover is removed.

47. The EKG recording accessory system of claim 34 in which said disposable precordial pads of the system further includes six embedded electrodes in a predetermined geometry.

48. The EKG recording accessory system of claim 47 in which said disposable precordial pads of the system further includes four connection points for attachment of limb electrodes.

49. The EKG recording accessory system of claim 48 in which said disposable precordial pads of the system further includes two right heart electrodes.

50. The EKG recording accessory system of claim 34 in which said embedded electrodes in said disposable precordial pads of the system each have a micro-transmitter for wireless transmission of a signal to said data transmitting module.

51. The EKG recording accessory system of claim 34 in which said universal adaptor/receiver for an EKG recording and monitoring system further comprises an EKG machine interface, to which wire leads from any brand of EKG may be attached, and a precordial pad interface to which a single multi strand cable from said disposable precordial pads of the system may be attached.

52. The EKG recording accessory system of claim 51 in which said universal adaptor/receiver further comprises a receiver for receiving data from the micro-transmitter and transferring said data to the EKG recorder.

53. The EKG recording accessory system of claim 34 in which said universal adaptor/receiver further comprises a display to indicate body temperature.

54. The EKG recording accessory system of claim 34, in which said universal adaptor/receiver further includes a selection means to select between wired or wireless reception of signals.

55. The EKG recording accessory system of claim 34 in which said universal adaptor/receiver further includes an EKG machine selector function by which a specific EKG machine may be selected for interface with said data from said disposable precordial pads.

56. A disposable EKG precordial pad comprising:
a pad body for conductive attachment to a patient's torso, with a first and a second surface, with a plurality of electrodes for contact with a patient, and with an adhesive surface on said body surface for contact with said patient's skin; and
a positioning device for use with a patient for orienting and positioning said pad body for correct positioning on said patient, in which said positioning device comprises an anatomy feature positioner for determining a horizontal position of said pad body, and a medial positioner for determining a vertical orientation of said pad body.

57. The disposable EKG precordial pad of claim 56 in which said anatomy feature positioner comprises a notch consistent with a supra sternal notch of a patient, and which is designed to be placed on the edge of said supra sternal notch.

58. The disposable EKG precordial pad of claim 57, which further includes a pair of sternal clavicular nodes for placement over the sternal clavicular joints of the patient.

59. The disposable EKG precordial pad of claim 56, which further includes an umbilicus extension as said medial positioner to allow placement of said precordial pad in line with a patient's umbilicus, for use in placement of said precordial pad for correct vertical orientation.

60. A disposable EKG precordial pad comprising:
a pad body for conductive attachment to a patient's torso with a first and a second surface, with a plurality of electrodes for contact with a patient, and with an adhesive surface on said body surface for contact with said patient's skin;
a positioning device for use with a patient for orienting and positioning said pad body for correct positioning on said patient, in which said positioning device defines a notch consistent with a supra sternal notch of a patient, and which is designed to be placed on the edge of said supra sternal notch;
a pair of sternal clavicular nodes, for placement over the sternal clavicular joints of the patient; and
an umbilicus extension for placement in line with a patient's umbilicus for use in placement of said precordial pad.

61. The disposable EKG precordial pad of claim 60, which further includes a sizing strategy that comprises using a selection of different size pads for different sizes of patients.

62. The disposable EKG precordial pad of claim 61 in which said sizing strategy is based on chest girth of a patient.

63. The disposable EKG precordial pad of claim 56, in which said pad body includes four limbs leads, in addition to EKG pads, and said pad body defines a left arc and a right arc sized to allow the EKG pad of the invention to be positioned between the breast of a female patient, with said left arc adjacent said patient's left breast, and said right arc adjacent said patient's right breast.

64. The disposable EKG precordial pad of claim 56, in which said pad body includes four limbs leads in addition to EKG pads, and said pad body defines a left arc and a right arc sized to allow the EKG pad of the invention to be positioned between the breast of a female patient, with said left arc adjacent said patient's left breast, and said right arc adjacent said patient's right breast;
a positioning device for use with a patient for orienting and positioning said pad body for correct positioning on said patient in which said positioning device defines a notch consistent with a supra sternal notch of a patient, and which is designed to be placed on the edge of said supra sternal notch;
a pair of sternal clavicular nodes, for placement over the sternal clavicular joints of the patient; and
an umbilicus extension for placement in line with a patient's umbilicus, for use in placement of said precordial pad.

65. The disposable EKG precordial pad of claim 56, in which said pad body includes a plurality of EKG electrodes on flexible stalks extending from said pad body, which allow said electrodes to be placed on a portion of a female patient's breast, while said pad body is placed on said patient's chest wall.

66. The disposable EKG precordial pad of claim 56, in which said pad body includes a plurality of EKG electrodes on flexible stalks extending from said pad body, which allow said electrodes to be placed on a portion of a female patient's breast while said pad body is placed on a patient's chest wall;
four limbs leads, in addition to EKG pads;
in which said pad body defines a left arc and a right arc sized to allow the EKG pad of the invention to be positioned between the breast of a female patient, with said left arc adjacent said patient's left breast, and said right arc adjacent said patient's right breast.

67. A disposable EKG precordial pad comprising:
a pad body for conductive attachment to a patient's torso with a first and a second surface, with a plurality of electrodes for contact with a patient, and with an adhesive surface on said body surface for contact with said patient's skin;
a positioning device for use with a patient for orienting and positioning said pad body for correct positioning on said patient in which said positioning device defines a notch consistent with a supra sternal notch of a patient, and which is designed to be placed on the edge of said supra sternal notch;
a pair of sternal clavicular nodes for placement over the sternal clavicular joints of the patient;
an umbilicus extension for placement in line with a patient's umbilicus for use in placement of said precordial pad;
a plurality of EKG electrodes on flexible stalks extending from said pad body, which allow said electrodes to be placed on a portion of a female patient's breast while said pad body is placed on a patient's chest wall;
four limbs leads, in addition to EKG pads;
in which said pad body defines a left arc and a right arc, sized to allow the EKG pad of the invention to be positioned between the breast of a female patient, with said left arc adjacent said patient's left breast, and said right arc adjacent said patient's right breast,
a pad body for conductive attachment to a patient's torso with a first and a second surface, with a plurality of electrodes for contact with a patient, and with an adhesive surface on said body surface for contact with said patient's skin; and
a positioning device for use with a patient for orienting and positioning said pad body for correct positioning on said patient in which said positioning device defines a notch consistent with a supra sternal notch of a patient, and which is designed to be placed on the edge of said supra sternal notch.

68. The disposable EKG precordial pad of claim 67, which further includes a pair of sternal clavicular nodes for placement over the sternal clavicular joints of the patient.

69. The disposable EKG precordial pad of claim 67, which further includes an extension for placement in line with a patient's umbilicus, for use in placement of said precordial pad.

70. The disposable EKG precordial pad of claim 67, which further includes electrodes that are embedded in said pad body but which extend from said pad body before application for electrical connection to the patient's torso.

71. The disposable EKG precordial pad of claim 67, which further includes a data transmitting module for sending a plurality of signals from said embedded electrodes to an EKG machine.

72. The disposable EKG precordial pad of claim 67 which further includes a temperature sensor for sensing the temperature of said patient's skin, and for sending temperature information to said EKG machine.

73. The precordial pad of claim 67 in which the data-transmitting module is a wireless transmitter.

74. The precordial pad of claim 67 in which the data-transmitting module is a wire cable with individual wires.

75. The disposable EKG precordial pad of claim 67 in which said plurality of electrodes includes one or more micro-transmitters for sending a signal from said electrode.

76. The disposable EKG precordial pad of claim 67 which further includes a circuit layer which is located between said first and said second layer, and which includes electronic connections between said electrodes and said data transmitting module.

77. The disposable EKG precordial pad of claim 76 in which said circuit layer is comprised of an insulating sheet on which is placed electrical connections in the form of conductive pathways.

78. The disposable EKG precordial pad of claim 77 in which said conductive pathways are metallic ink circuitry.

79. The precordial pad of claim 67, which includes an adhesive cover, which is removable for exposing said adhesive surface before use.

80. The precordial pad of claim 67 in which said electrodes include an electrode extension device for causing said electrodes to extend beyond said adhesive surface when said adhesive cover is removed.

81. The precordial pad of claim 80 in which said electrode extension device is a biased member mounted between said electrode and said pad, which is held in biased position by said adhesive cover, and which moves said electrode away from said pad when said adhesive cover is removed.

82. The precordial pad of claim 80 in which said electrode extension device is a spring.

83. The precordial pad of claim 80 in which said electrode extension device member is a foam structure.

84. The precordial pad of claim 67 in which said electrode is packaged under said adhesive cover with a layer of transmitting gel so that when said adhesive cover is removed, said transmitting gel and said embedded electrodes are configured to be exposed.

85. The precordial pad of claim 67 in which said thermometer sensor is a low reading thermometer sensor.

86. The precordial pad of claim 67 that further comprises six precordial electrodes in a predetermined geometry.

87. The precordial pad of claim 86, which further includes connection points for attachment of limb electrodes.

88. The precordial pad of claim 87, which further includes connection points for attachment of four limb electrodes at the user's discretion.

89. The precordial pad of claim 67 that further comprises six torso electrodes and four limb electrodes built into a single precordial pad.

90. The precordial pad of claim 89, which further includes two additional electrodes for right heart monitoring.

91. The precordial pad of claim 67 in which said embedded electrodes each have a micro-transmitter for wireless transmission of a signal to said data transmitting module.

92. The precordial pad of claim 67 in which said signal export module further includes a temperature display for indicating the patient's body temperature.

93. The precordial pad of claim 67 in which the signal export module is a wireless transmitter.

94. The precordial pad of claim 67 in which the data-transmitting module is a wire harness for connection to an EKG machine.

95. The precordial pad of claim 67 that further includes a low temperature window for displaying patient temperature information.

96. The precordial pad of claim 67 in which said pad further includes the capability to connect to non-EKG devices including defibrillators, real time heart monitors and external pacemakers.
